(19) United States
(12) Patent Application Publication
(10) Pub. No.: US 2014/0323794 A1
(43) Pub. Date: Oct. 30, 2014

(54) ELECTRONIC BRACHYTHERAPY RADIATION APPLICATION APPARATUS COMPRISING A PIEZOELECTRICALLY POWERED X-RAY SOURCE

(71) Applicant: KONINKLIJKE PHILIPS N.V., EINDHOVEN (NL)

(72) Inventors: Carolina Ribbing, Aachen (DE); Gereon Vogtmeier, Aachen (DE)

(73) Assignee: KONINKLIJKE PHILIPS N.V., EINDHOVEN (NL)

(21) Appl. No.: 14/357,692

(22) PCT Filed: Nov. 9, 2012
(86) PCT No.: PCT/IB2012/056300

§ 371 Date: May 13, 2014

Related U.S. Application Data
(60) Provisional application No. 61/559,766, filed on Nov. 15, 2011.

(51) Int. Cl. A61N 5/10

(52) U.S. Cl. CPC A61N 5/1001; A61N 2005/1022
USPC 600/3

(57) ABSTRACT

The invention relates to a radiation application apparatus for applying radiation at a location within an object. The radiation application apparatus comprises a transforming unit (2) for being arranged within the object at the location and for transforming ultrasound energy to electrical energy, and a radiation source (4) for being arranged within the object and for generating radiation (5) to be applied at the location within the object, wherein the radiation source (4) is driven by the electrical energy. Since the transforming unit transforms the ultrasound energy to electrical energy being used by the radiation source, it is not necessary to transfer electrical energy to the radiation source, i.e., for example, corresponding cables, which may have to be isolated, are not necessarily required. Insulation problems and corresponding safety problems, which may be present, if cables, in particular, corresponding high voltage cables, are used, can therefore be reduced.

### FIELD OF THE INVENTION

The invention relates to a radiation application apparatus and a radiation application method for applying radiation at a location within an object.

### BACKGROUND OF THE INVENTION

In electronic brachytherapy a miniature x-ray tube is navigated to a desired location within a person, at which x-rays are to be applied, for example, for treating a tumor, wherein the x-ray tube is operated at a voltage of, for instance, 50 kV. Since this high voltage has to be transferred from the outside of the person to the x-ray tube within the person, for safety reasons extreme requirements have to be applied to cable insulation and current limitation reliability in case of cable fracture or short circuit.

### SUMMARY OF THE INVENTION

It is an object of the present invention to provide a radiation application apparatus and a radiation application method for applying radiation at a location within an object, wherein requirements regarding electrical insulation and current limitation reliability can be reduced.

In a first aspect of the present invention a radiation application apparatus for applying radiation at a location within an object is presented, wherein the radiation application apparatus comprises:

a transforming unit for being arranged within the object at the location and for transforming ultrasound energy to electrical energy,

a radiation source for being arranged within the object and for generating radiation to be applied at the location within the object, wherein the radiation source is driven by the electrical energy.

Since the transforming unit transforms the ultrasound energy to electrical energy, which is used by the radiation source for generating the radiation, it is not necessary to transfer electrical energy to the radiation source, i.e., for example, corresponding cables, which may have to be isolated, are not necessarily required. Since electrical energy does not need to be transferred to the radiation source, insulation problems and corresponding safety problems, which may be present, if cables, in particular, corresponding high voltage cables, are used, can be reduced.

The object is preferentially a person, wherein the radiation is applied to an inner part of the person. The object can also be an animal or a technical object. Specifically, the radiation can be applied to, for example, a tumor within a person, to which radiation has to be applied for destroying the tumor.

It is preferred that the radiation source is an x-ray source for generating x-rays as radiation, when the electrical energy is applied to the x-ray source. The x-ray source is preferentially a miniature x-ray source for being arranged within, for example, a brachytherapy applicator comprising a catheter or a needle. Thus, for example, tissue within a person can be treated by using x-rays. Typical operating voltages lie preferentially in the range of 20 to 100 kV, which allow producing x-ray spectra with mm to cm range in tissue. The operating current being the tube current can be chosen so as to produce a high-enough dose rate at the typical target tissue. In particular, the operating current can be in the range of 20 to 800 μA.

The transforming unit is preferentially adapted to transform the ultrasound energy into high voltage for providing electrical energy. The high voltage is preferentially within a range needed for driving an x-ray tube. It is preferentially in the range of 20 kV to 100 kV and further preferred in the range of 40 kV to 100 kV.

The transforming unit can be adapted to provide pulsed electrical energy, wherein the radiation source can be adapted to generate pulsed radiation based on the pulsed electrical energy. In particular, the transforming unit can be adapted to generate a pulsed electric field between an anode and a cathode of an x-ray tube and, thus, a pulsed current, which may be in the kHz to MHz frequency range. In another embodiment, the transforming unit can also be adapted to provide continuous electrical energy and the radiation source can be adapted to generate continuous radiation based on the continuous electrical energy.

It is further preferred that the transforming unit comprises a piezoelectric element for transforming the ultrasound energy into electrical energy. This allows transforming the ultrasound energy into electrical energy by generating a corresponding voltage in a relatively simple way. The piezoelectric element can be incorporated in or near the radiation source. In particular, the piezoelectric element can be situated behind the cathode or behind the anode of a miniature x-ray source, in order to produce an accelerating electric field between the cathode and the anode, while still not being hit by electrons between the two electrodes. The piezoelectric element can also be integrated with the anode or the cathode. In particular, it can be identical with the cathode of the x-ray source. In this case, electrons are emitted from the surface of the piezoelectric element when the electric field in the piezoelectric element is reversed, producing a pulsed electron emission. This allows reducing the size of the radiation source, thereby facilitating an introduction of the radiation source and the transforming unit into the object.

The piezoelectric element is preferentially ceramic and may comprise at least one of the following materials: lead zirconate titanate (PZT), quartz, LiNbO₃, LiTaO₃, GaPO₄, La₃Ga₅SiO₁₄, BaTiO₃, KNbO₃, Na₂WO₃, Ba₂NaNb₅O₅, Pb₂KNb₅O₁₅, NaKNb, BiFeO₃, NaNbO₃. Polymeric piezoelectrica may also be used like polyvinylidene fluoride (PVDF), which exhibits significantly higher piezoelectricity than quartz.

The transforming unit can comprise several piezoelectric elements for transforming the ultrasound energy into voltage for providing electrical energy, wherein the piezoelectric elements can be arranged such that the voltages of the several piezoelectric elements are combined to a combined voltage being larger than each voltage produced by a respective single piezoelectric element. For example, the piezoelectric elements, which may be thin films or slabs, can be geometrically arranged such that the electric fields and, thus, the voltages generated by the several piezoelectric elements are amplified, in particular, added up, to a combined electric field and, thus, to the combined voltage. This allows generating relatively high voltages for providing the electrical energy.

In a preferred embodiment, the object is a person and the transforming unit and the radiation source are configured to be arrangable within the person for applying the radiation at the location within the person by using an applicator. In particular, the applicator can comprise a casing which is preferentially tube-like. It can be an interstitial tube like a catheter or a needle used in interventional procedures. The transforming unit and the radiation source can be arrangable within the casing and the casing can be adapted to be introduced into the object for applying the radiation at the location within the object. The transforming unit and the radiation source can therefore be arranged within the object by arranging the casing, in which the transforming unit and the radiation source can be located, within the object. The outside of the casing is preferentially adapted such that it does not damage the object, when introduced into the same. The transforming unit and the radiation source can therefore be forwarded into the object, for example, without harming inner parts of the object by outer parts of the transforming unit and the radiation source.

The applicator can be regarded as being a part of the radiation application apparatus or it can be regarded as being a separate element. The applicator is preferentially a brachytherapy applicator like a balloon applicator, a vaginal applicator, or a SAVI-type applicator.

It is further preferred that the radiation application apparatus comprises an electrical energy storing unit for storing the electrical energy and for providing the stored electrical energy to the radiation source. The electrical energy storing unit is preferentially a battery. For instance, the electrical energy storing unit can be a thin-film battery or a capacitor. It is also preferred that the radiation application apparatus comprises an amplification unit for increasing the generated voltage, before being used by the radiation source for generating the radiation. The amplification unit comprises, for example, a transformer and/or a voltage multiplier. The voltage multiplier is, for example, a Villard cascade.

The radiation application apparatus comprises preferentially an ultrasound energy generating device for generating the ultrasound energy to be transformed by the transforming unit to electrical energy. The ultrasound energy generating device can be adapted to produce continuous or pulsed ultrasound waves for generating corresponding radiation, i.e. preferentially corresponding x-rays.

In an embodiment, the radiation application apparatus further comprises an ultrasound transferring unit for transferring ultrasound waves from the outside of the object to the transforming unit. The ultrasound transferring unit is preferentially an ultrasound transferring cable or an ultrasound transferring tube for transferring ultrasound waves from an ultrasound energy generating device to the transforming unit. The ultrasound transferring unit is preferentially at least partly arrangable within a casing like a tube arranged with the object, in order to transfer ultrasound from the outside of the object via the ultrasound transferring unit through the casing to the transforming unit within the object. Transferring the ultrasound energy to the transforming unit via the ultrasound transferring unit can lead to an improvement of the efficiency of generating the radiation with respect to the generated ultrasound energy.

In another embodiment, the transforming unit can be adapted to receive ultrasound waves wirelessly.

The ultrasound energy generating device is preferentially adapted to be arranged ex vivo. It can be a separate unit or it can be a part of an ultrasound imaging system. In the latter case, a general-purpose sonographic device in combination with an additional ultrasound transducer for providing the ultrasound energy to be transformed by the transforming unit can be used, wherein the additional ultrasound transducer and the transforming unit can be connected to opposite ends of an ultrasound transferring cable for transferring the ultrasound energy from the additional ultrasound transducer to the transforming unit. The ultrasound imaging system, or other another imaging modality, can be used for placing the radiation source and/or an applicator.

The ultrasound energy generating device can be adapted to a) send the ultrasound energy to the object, b) receive reflected ultrasound energy from the object, c) determine the position of the transforming unit within the object from the received reflected ultrasound energy, and d) focus the send ultrasound energy onto the determined position of the transforming unit. For instance, the ultrasound energy generating device can be adapted to calculate the position of the transforming unit within the object by using known radar-like position determination algorithms. The focusing of the ultrasound energy to the position of the transforming unit allows further increasing the efficiency of generating radiation within the object based on generated ultrasound energy.

The object is preferentially a person, wherein the transforming unit, the radiation source, and the ultrasound energy generating device can be configured to be arrangable within the person for applying the radiation at the location within the person by using the above mentioned applicator. This allows placing the ultrasound generating unit close to the transforming unit within the person, thereby further enhancing the ultrasound power transmission to the transforming unit.

The ultrasound generating unit can comprise a set of ultrasound transducers placed in an array in an x-y plane, generating an ultrasound beam in a z direction. In the wireless case, the ultrasound waves may be focused to the piezoelectric element by means of, for example, ultrasound focusing devices as generally used in, for example, high-intensity focused ultrasound (HIFU) therapy. The ultrasound beam can be focused (a) geometrically, for example, with a lens or with a curved transducer, or (b) electronically, using a so-called phased array, where the relative phases of elements in a transducer array are adjusted to steer the beam to various locations. Using the phased-array technique, the focus can be moved in the object, so as to power an radiation application generating unit like an x-ray source placed at various locations within the object or even to follow the source path in an applicator in real-time.

The radiation application apparatus is preferentially adapted to be used for interventional radiotherapy, wherein the radiation is provided by x-rays which are applied to the object. In particular, the radiation application apparatus is preferentially adapted for being used in brachytherapy.

In a further aspect of the present invention an ultrasound energy generating device for generating ultrasound energy is presented, wherein the ultrasound energy generating device is adapted to cooperate with the radiation application apparatus as defined in claim 1 for applying radiation at a location within an object, wherein the ultrasound energy generating device is adapted to generate ultrasound energy, which is transformable by the transforming unit to electrical energy for driving the radiation source to generate radiation to be applied within the object.

In a further aspect of the present invention a radiation application method for applying radiation at a location within an object is presented, wherein the radiation application method comprises:

arranging a transforming unit and a radiation source at the location within the object,

generating ultrasound energy by an ultrasound energy generating device,

transforming the ultrasound energy to electrical energy by the transforming unit within the object,

generating radiation to be applied at the location within the object by the radiation source, wherein the radiation source is driven by the electrical energy.

It shall be understood that the radiation application apparatus of claim 1, the ultrasound energy generating device of claim 14 and the radiation application method of claim 15 have similar and/or identical preferred embodiments, in particular, as defined in the dependent claims.

It shall be understood that a preferred embodiment of the invention can also be any combination of the dependent claims with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of a radiation application apparatus for applying radiation to an object,

FIG. 2 shows schematically and exemplarily an embodiment of an arrangement of a transforming unit and a radiation source within a catheter,

FIG. 3 shows schematically and exemplarily an embodiment of an ultrasound energy generating device on a person,

FIGS. 4 to 6 show exemplarily and schematically different embodiments of possible arrangements of a transforming unit and a radiation source within the catheter,

FIGS. 7 and 8 show schematically and exemplarily arrangements of several piezoelectric elements of a transforming unit,

FIG. 9 shows schematically and exemplarily a transversally acting piezoelectric element of a transforming unit,

FIG. 10 shows exemplarily and schematically a further embodiment of a possible arrangement of a transforming unit and a radiation source within the catheter,

FIG. 11 shows schematically and exemplarily an embodiment of an arrangement of a radiation source, a transforming unit and an ultrasound energy transferring unit within a catheter,

FIG. 12 shows schematically and exemplarily an embodiment of an arrangement of a radiation source, a transforming unit and an ultrasound energy generating device within a catheter,

FIG. 13 shows exemplarily and schematically a further embodiment of a possible arrangement of a transforming unit and a radiation source within the catheter,

FIG. 14 shows a piezoelectric element for generating a voltage and a high voltage cascade for amplifying the voltage, and

FIG. 15 shows a flowchart exemplarily illustrating a radiation application method for applying radiation to an object.

### DETAILED DESCRIPTION OF EMBODIMENTS

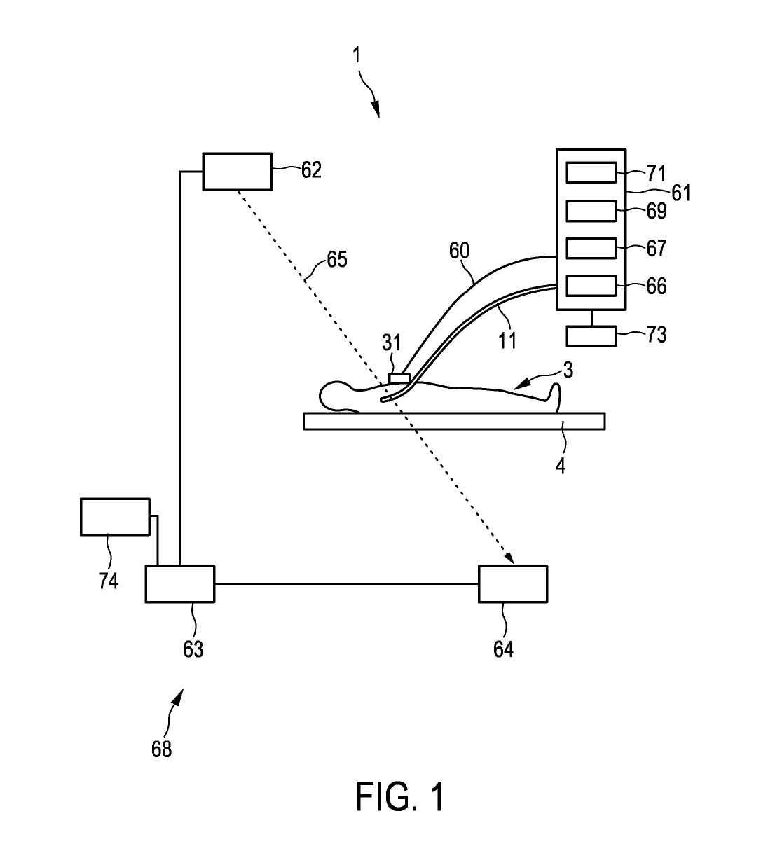

FIG. 1 shows schematically and exemplarily an embodiment of a radiation application apparatus 1 for applying radiation at a location within an object 3. In this embodiment, the object 3 is a person located on a person table 4. The radiation application apparatus 1 comprises a catheter navigation unit 66 for navigating a catheter 11 being used as an applicator for applying a radiation source within a person to a desired location within the person 3. After the catheter 11 has been navigated to the desired location, a radiation source for generating radiation to be applied at the location within the person and a transforming unit for transforming ultrasound energy to electrical energy, which is used for driving the radiation source, are introduced into the person 3 via the catheter 11. For forwarding and retracting the radiation source and the transforming unit within the catheter 11, the transforming unit and the radiation source are connected via, for example, a wire to a motor 67. After the transforming unit and the radiation source have been moved to a desired location within the catheter 11, the radiation can be applied to, for example, a tumor within the person for destroying the tumor. In particular, the radiation source can be inserted into a tumor cavity or in a natural lumen, in order to apply the radiation at and/or close to these places.

The radiation source is an x-ray source for generating x-rays, while the electrical energy is applied to the x-ray source. In this embodiment, the x-ray source is a miniature x-ray source that can be arranged within the catheter. The x-ray source and the transforming unit are configured to transform the ultrasound energy to the electrical energy such that an electrical field is produced in a region between an anode and a cathode of the x-ray source, in order to accelerate electrons emitted from the cathode towards the anode.

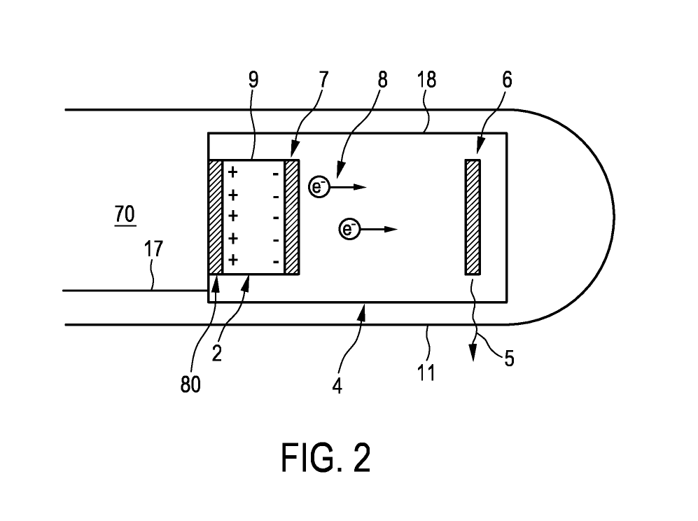

FIG. 2 shows schematically and exemplarily an x-ray source 4 and a transforming unit 2 within the catheter 11. The x-ray source 4 comprises a cathode 7 and an anode 6 for accelerating electrons 8 towards the anode 6. When the electrons 8 are incident on the anode 6, x-rays 5 are generated in a known way. Optionally, a dielectric can be present between the anode 6 and the cathode 7 (not shown in FIG. 2).

The transforming unit 2 comprises a piezoelectric element 9 for transforming the ultrasound energy into electrical energy. The piezoelectric element can be regarded as being a piezoelectric receiver for receiving ultrasound waves, i.e. the ultrasound energy, and for generating a voltage depending on the received ultrasound waves. When irradiated with ultrasound energy, the piezoelectric receiver is alternatingly compressed and expanded, producing an electric field, i.e. a voltage. In this embodiment, the piezoelectric element 9 is integrated with the cathode 7 and can be regarded as being identical with the cathode 7 of the x-ray source 4. The electrons 8 are emitted from the surface of the piezoelectric element 9, when the electric field in the piezoelectric element 9 is reversed, producing a pulsed electron emission. In particular, the piezoelectric element 9 can be pressed together by the received ultrasound waves, thereby polarizing the piezoelectric element. In the electrodes compensation charges are then collected, which compensate the polarization of the piezoelectric element. For instance, a positive polarization on a surface of the piezoelectric element is compensated by electrons in the electrode covering this surface of the piezoelectric element. If the polarization in the piezoelectric element is reversed, electrons are emitted from the electrode.

In an embodiment, between the cathode and the anode low pressure gas can be present, wherein the electrical field generated by the piezoelectric element can generate ions, which are accelerated and interact with gas atoms to create further electrons and positive ions. The positive ions can be accelerated towards the cathode, thereby knocking further electrons out of the cathode. The generated electrons are accelerated towards the anode, where the x-rays are generated. X-rays can also be generated in the cathode by the impinging ions.

The transforming unit 2 and the radiation source 4 are arranged within a grounded housing 18, which can be vacuum-tight and to which a moving wire 17 is attached for moving the housing 18 within the catheter 11. The anode 6 and a further electrode 80 on the surface being opposite to the cathode 7 are preferentially connected with ground by electrically connecting the anode 6 and the further electrode 80 with the housing 18. The moving wire 17 is also connected with the motor 67 for allowing the motor 67 to move the transforming unit 2 and the radiation source 4 within the catheter 11. This moving of the transforming unit and the radiation source can be performed automatically, semi-automatically or manually. In an embodiment, the transforming unit and the radiation source are moved via the moving wire by hand only, without using the motor 67. A cooling fluid 70 is provided within the catheter 11 by a cooling fluid providing unit 69. The cooling fluid 70 cools the radiation source 4 and can also act as a medium for transferring ultrasound waves from the outside of the person 3 to the transforming unit 2.

Referring again to FIG. 1, the radiation application apparatus further comprises an ultrasound energy generating device 31 for generating ultrasound energy to be transformed by the transforming unit 2 to electrical energy. The ultrasound energy generating device 31 can be adapted to produce continuous or pulsed ultrasound waves for generating corresponding radiation, i.e. corresponding x-rays. The ultrasound energy generating device 31 is arranged on the outer skin of the person 3. The coupling of the ultrasound energy can be enhanced by use of, for example, an index matching gel or other liquid as known from ultrasound imaging, where a hand-held transducer is placed directly on and moved over the outer skin.

In this embodiment, the ultrasound energy is transmitted wirelessly to the transforming unit 2 from the outside of the person 3 to the inside location, where the x-rays 5 are to be applied. The ultrasound energy generating device 31 is connected via an electrical connection 60 like an electrical cable with an ultrasound control unit 71. The ultrasound control unit 71, the cooling fluid providing unit 69, the motor 67 and the catheter navigation unit 66 can be components of a radiation application apparatus control unit 61.

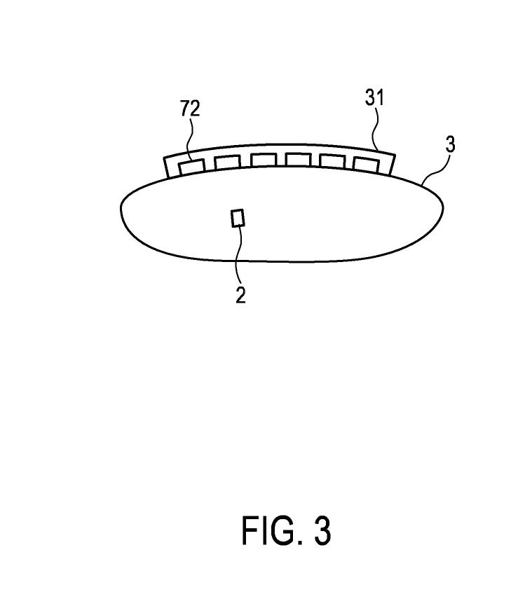

The ultrasound energy generating device 31, which is schematically and exemplarily shown in more detail in FIG. 3, comprises an array of ultrasound transducers 72 for sending ultrasound energy to the transforming unit 2 and for receiving reflected ultrasound energy from the transforming unit 2. The phases of the emission of the ultrasound waves from the different ultrasound transducers 72 can be controlled by the ultrasound control unit 71 such that the sent ultrasound energy is focused onto the transforming unit 2. In particular, the received reflected ultrasound energy from the transforming unit 2 can be used for determining the position of the transforming unit 2, wherein then the emission of the ultrasound energy can be controlled such that it is focused onto the determined position of the transforming unit 2. The position of the transforming unit 2 can be determined based on the time needed by an ultrasound wave to travel from the respective ultrasound transducer 72 to the transforming unit 2 and back from the transforming unit 2 to the respective ultrasound transducer 72. In an embodiment, for determining the position of the transforming unit 2 the ultrasound transducers 72 can be operated individually such that for each reflected ultrasound wave the origin of the ultrasound wave, i.e. which ultrasound transducer 72 has generated the ultrasound wave, is unambiguously known. Based on the determined times needed by the ultrasound waves for traveling to the transforming unit 2 and back to the ultrasound transducers 72 the position of the transforming unit 2 can be reliably determined. In other embodiments, the position of the transforming unit 2 can be determined in another way. For example, an x-ray fluoroscopy system 68 can be used for determining the position of the transforming unit 2. In FIG. 3, further elements like the radiation source 4 are not shown for clarity reasons.

Since a high voltage cable from the outside of the person to the x-ray source is not needed, safety requirements can be reduced. Moreover, in the prior art the stiffness of a high voltage cable can reduce the mobility of the x-ray source within the person. Thus, since in the described embodiments a high voltage cable is not needed, the mobility of the x-ray source, for instance, with respect to movements along curved path ways, within the person can be improved.

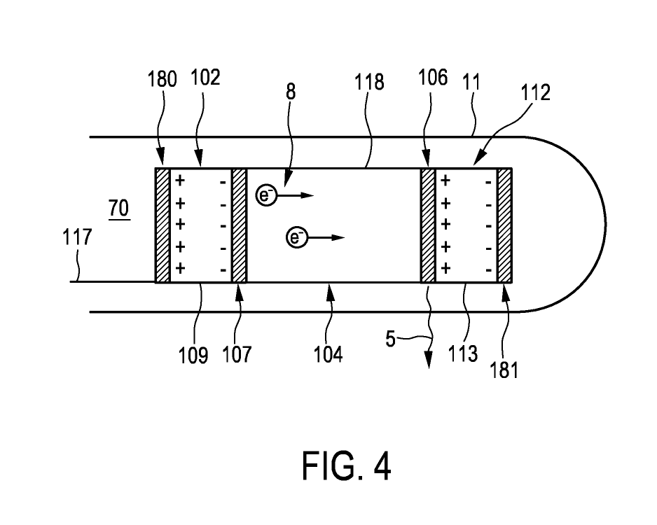

In another embodiment, the radiation source and the transforming unit can have another configuration within the catheter 11. For instance, as schematically and exemplarily shown in FIG. 4, the transforming unit 102, 112 can be distributed among the cathode 107 and the anode 106, i.e. a first piezoelectric element 109 of a first part of the transforming unit 102 can be integrated with the cathode 107 and a second piezoelectric element 113 of a second part 112 of the transforming unit can be integrated with the anode 106. The anode 106 and the cathode 107 are elements of an x-ray source 104, wherein, if ultrasound waves meet the piezoelectric elements, electrons 8 are generated and accelerated towards the anode 106, where the x-ray radiation 5 is generated. The transforming unit 102 and the radiation source 104 are arranged within a grounded housing 118, which can be a vacuum tight container, which can be moved within the catheter 11, i.e. the applicator, via a moving wire 117 by the motor 67. In the configuration exemplarily shown in FIG. 4 the ultrasound energy generating device can be adapted to generate ultrasound waves, which are configured such that the piezoelectric elements 109, 113, i.e. the electrodes 106, 107, are alternatingly used as cathode and anode, wherein x-rays are produced alternatingly at the electrodes 106, 107. A further electrode 180 of the piezoelectric element 109, which is opposite to the electrode 107, and a further electrode 181 of the piezoelectric element 113, which is opposite to the electrode 106, are grounded, i.e. preferentially they are electrically connected to the grounded housing 118. The electrodes 106, 107 are not grounded.

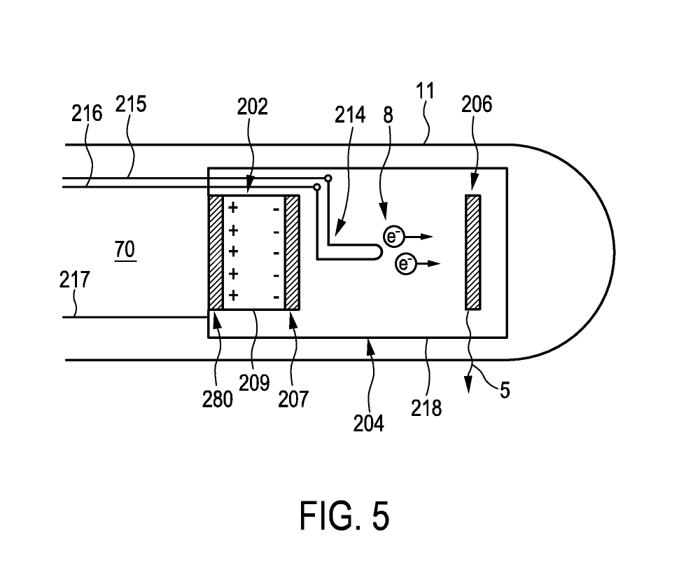

FIG. 5 shows schematically and exemplarily a further embodiment of an arrangement of a radiation source and a transforming unit within the catheter 11. In this embodiment, the transforming unit 202 comprises a piezoelectric element 209 integrated with a first electrode 207 of a radiation source 204. The radiation source 204 is an x-ray tube, wherein electrons 8 generated by a filament cathode 214 are accelerated between the first electrode 207 and an anode 206 towards the anode 206. The transforming unit 202 and the radiation source 204 are arranged within a grounded housing 218, which is movable within the catheter 11 by the motor 67 via a moving wire 217. The anode 206 and a further electrode 280 of the piezoelectric element 209 are grounded, and the filament cathode 214 is connected via electrical connections 215, 216 with an external voltage source such that the filament cathode 214 can emit electrons. The anode 206 and the further electrode 280 are preferentially electrically connected with the grounded housing 218.

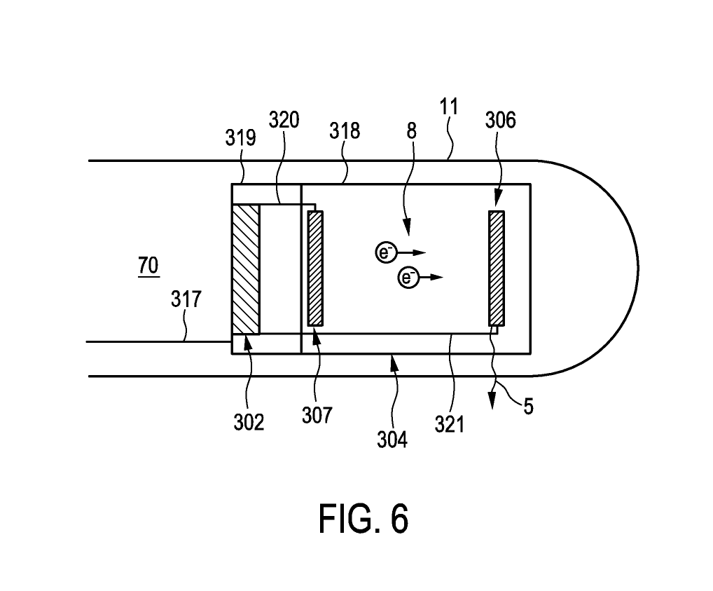

FIG. 6 shows schematically and exemplarily a further arrangement of a transforming unit and a radiation source within the catheter 11. In FIG. 6, the transforming unit 302 is not integrated with a cathode 307 or an anode 306 of an x-ray source 304. The transforming unit 302 is an element, which is separated from the cathode 307 and the anode 306 and connected with them via electrical connections 320, 321 for providing the electrical energy generated by the transforming unit 302 to the radiation source 304. In this embodiment, the transforming unit 302 is arranged within a housing 319 and the radiation source 304 is arranged within a further housing 318. The two housings 318, 319 can be moved together with the transforming unit 302 and the radiation source 304 by the motor 67 via a moving wire 317. In other embodiments, the transforming unit 302 and the radiation source 304 can also be located within a same housing. The transforming unit 302 is adapted to transform ultrasound energy into a corresponding voltage, which is applied to the cathode 307 and the anode 306 for accelerating the electrons 8 towards the anode 306.

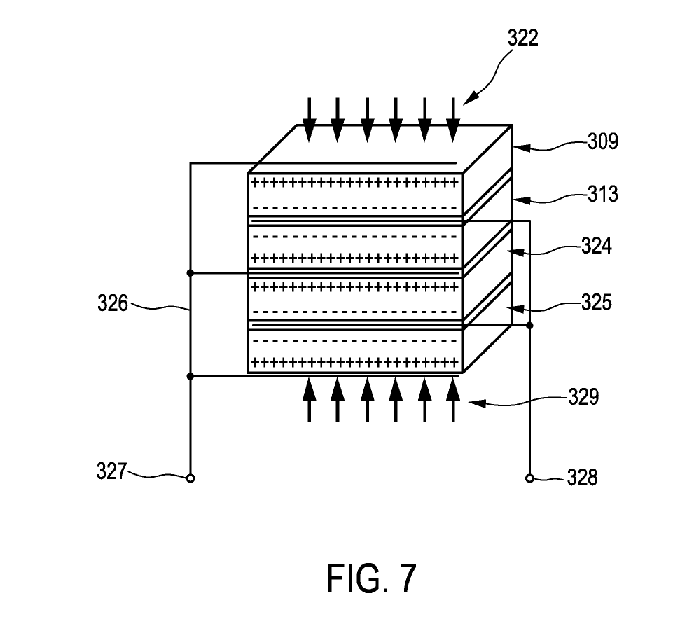

The transforming unit 302 can comprise several piezoelectric elements 309, 313, 324, 325 as schematically and exemplarily shown in FIG. 7. In FIG. 7, the piezoelectric elements 309, 313, 324, 325 are arranged such and the respective surfaces of the piezoelectric elements 309, 313, 324, 325 are electrically connected via electrical connections 326 such that voltages of the several piezoelectric elements 309, 313, 324, 325 are combined to a combined voltage being larger than each voltage produced by a respective single piezoelectric element. The resulting combined voltage is present between points 327, 328, which are connected with the cathode 307 and the anode 306 of the radiation source 304. In FIG. 7, the direction of the forces generated by the ultrasound waves are indicated by arrows 322, 323.

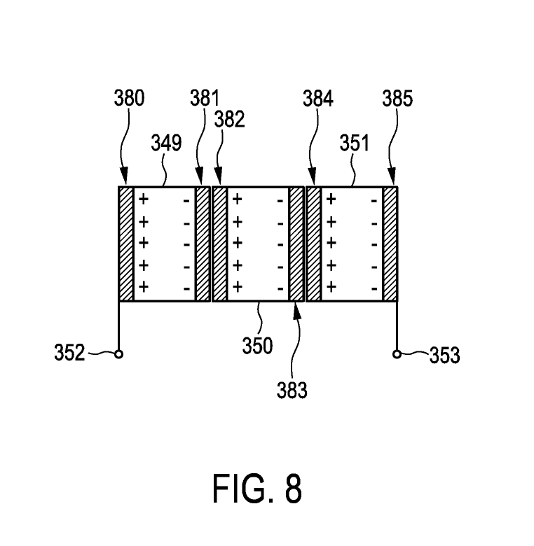

FIG. 8 shows schematically and exemplarily a further embodiment of an arrangement of piezoelectric elements 349, 350, 351 with electrodes 380 . . . 385, which may form the transforming unit 302 shown in FIG. 6. In this embodiment, the piezoelectric elements 349, 350, 351 are arranged such that voltages generated by the piezoelectric elements are added up to a combined voltage. Each piezoelectric element 349, 350, 351 can be a thin film or a slab of piezoelectric material. The resulting combined voltage can be provided to the anode 306 and the cathode 307 of the radiation source 304 via electrical connections 352, 353.

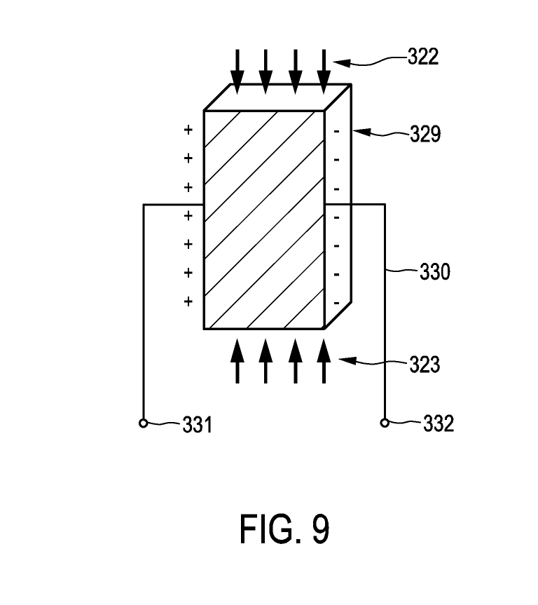

The respective piezoelectric element can be a longitudinally acting piezoelectric element or a transversally acting piezoelectric element. A transversally acting piezoelectric element is schematically and exemplarily shown in FIG. 9. In FIG. 9, the direction of the forces 322, 323 acting on the piezoelectric element 329 is transversal to the direction of the generated field, i.e. in FIG. 9 the force direction is vertically and the field direction is horizontally. The resulting voltage is provided between the points 331, 332 of the electrical connection 330 of the piezoelectric element 329. The anode and the cathode of the radiation source can be electrically connected to these points 331, 332 for providing the electrical energy generated by the piezoelectric element 329 to the x-ray source.

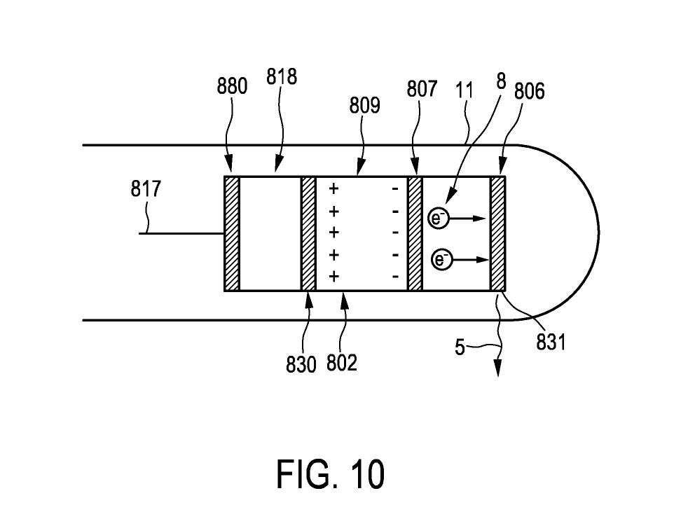

FIG. 10 shows schematically and exemplarily a further arrangement of a transforming unit 802 comprising a piezoelectric element 809. The piezoelectric element 809 is integrated with a cathode 807, wherein electrons 8 are accelerated between the cathode 807 and an anode 806, where x-ray radiation 5 is generated. Opposite to the cathode 807 a further electrode 830 is provided on the piezoelectric element 809 for generating an electric field between this electrode 830 and a further opposing electrode 880. The electrodes are located in a housing 818, which is moveable within the catheter 11 via a moving wire 817. The electrodes 880, 806 are grounded, in particular, by electrically connecting these electrodes 880, 806 with the grounded housing 818. In the situation shown in FIG. 10, the electrons 8 are accelerated towards the anode 806. Positive ions can be accelerated from the electrode 830 to the electrode 880. Depending on the received ultrasound waves, in a following situation it can be reverse. In both outer electrodes 806, 880 x-rays are generated.

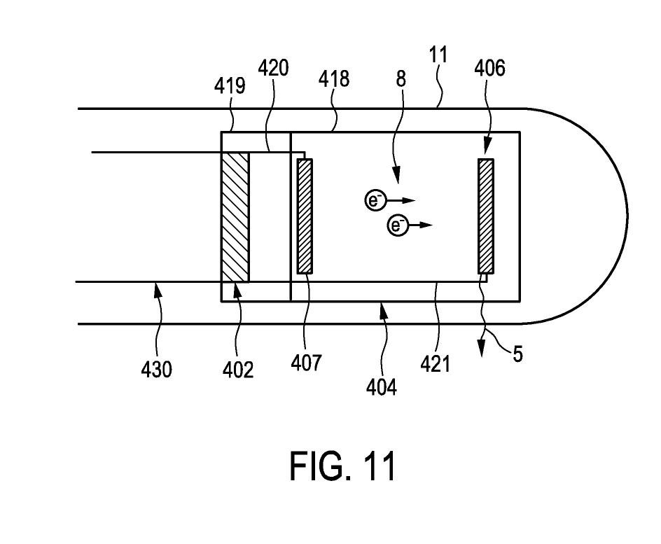

FIG. 11 shows schematically and exemplarily a further possible embodiment of an arrangement of a radiation source and a transforming unit within the catheter 11. In this embodiment, an ultrasound transferring unit 430 is located within the catheter 11 for transferring ultrasound waves from the outside of the person 3 to the transforming unit 402. The ultrasound transferring unit is preferentially an ultrasound transferring cable like one or several of the cables disclosed in U.S. Pat. No. 5,380,274 or in WO 90/01300. In this embodiment, outside of the person 3 the ultrasound transferring unit 430 is connected with an ultrasound energy generating device, i.e. with an ultrasound transducer, for transferring the generated ultrasound energy from the ultrasound energy generating device outside of the person to the transforming unit 402 inside of the person. The transforming unit 402 comprises at least one piezoelectric element for transforming the transferred ultrasound energy into electrical energy, in particular, into electrical voltage, wherein the voltage is applied to a cathode 407 and an anode 406 of an x-ray source 404 via electrical connections 420, 421. If the generated electrical voltage is applied to the cathode 407 and the anode 406, electrons 8 are generated and accelerated towards the anode 406, where x-rays 5 are produced. The radiation source 404 is arranged within a housing 418 and the transforming unit 402 is arranged within a housing 419. These housings with the transforming unit 402 and the radiation source 404 can be moved within the catheter 11 via the ultrasound transferring unit 430 or via a moving wire (not shown in FIG. 11) by using, for instance, an external motor.

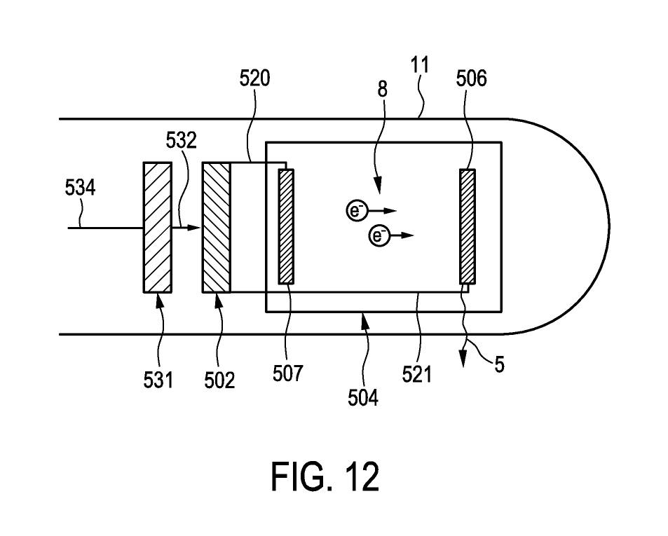

FIG. 12 shows schematically and exemplarily a further embodiment of an arrangement of a transforming unit and a radiation unit within the catheter 11. In FIG. 12, an ultrasound energy generating device 531 like a corresponding ultrasound transducer is arranged within the catheter 11. In another embodiment, the ultrasound energy generating device can also be provided within the person 3 close to the transforming unit by using another applicator, for example, another catheter.

The ultrasound generating energy device 531 is driven by an external voltage source (not shown in FIG. 12) via an electrical connection 534 for controlling the generation of ultrasound waves 532 within the person by the ultrasound energy generating device 531 from the outside of the person by the external voltage source. The ultrasound waves 532 are transformed into electrical voltage by the transforming unit 502, which comprises one or several piezoelectric elements. The electrical voltage is applied to a cathode 507 and an anode 506 via electrical connections 520, 521 for generating electrons 8 and accelerating the electrons 8 towards the anode 506, where x-rays 5 are generated.

The ultrasound energy generating device 531 and the transforming unit 502 can be separate elements as shown in FIG. 12, or they can be integrated for forming a high voltage generator. For instance, an integrated high-voltage generator including a piezoelectric element and an ultrasound actuator can be provided by micro electro mechanical systems (MEMS) technology.

If also the ultrasound energy generating device is located within the catheter, only a relatively low voltage has to be transferred into the person 3 for driving the ultrasound energy generating device. It is not necessary to use, for example, a high voltage cable for transferring high voltage to the location where the radiation is to be applied.

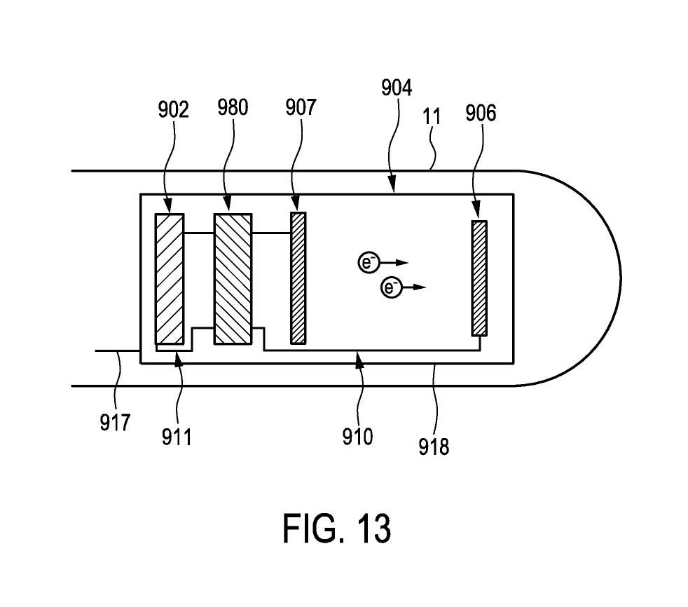

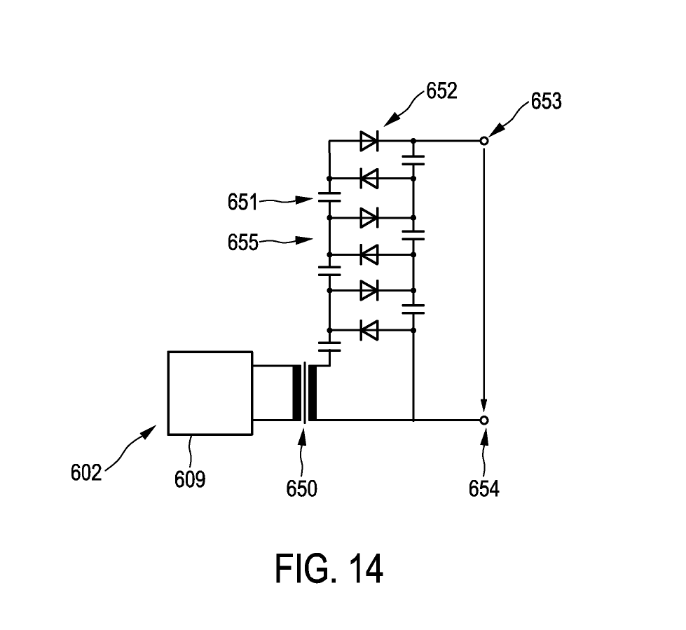

In a further embodiment, between the transforming unit and the radiation source a further unit for processing the electrical energy before being provided to the radiation source is present. For instance, as schematically and exemplarily shown in FIG. 13, between the transforming unit 902 comprising a piezoelectric element and the radiation source 904 with the cathode 907 and the anode 906 a processing unit 980 for processing the electrical energy can be provided. The processing unit 980 is, for example, an electrical energy storing unit, an amplification unit, a rectifier, et cetera. The processing unit 980 can be adapted to provide constant electrical energy to the radiation source for a certain time, in order to allow the radiation source to emit continuous radiation over a certain time. The transforming unit 902 is electrically connected with the processing unit 980 via electrical connections 911 and the processing unit 980 is electrically connected with the cathode 907 and the anode 906 via electrical connections 910. The housing 918 can be moved within the catheter 11 via a moving wire 917. The processing unit 980 can comprise one or several of the storing unit, the amplification unit, the rectifier, et cetera. If the processing unit 980 comprises an electrical energy storing unit for storing the electrical energy and for providing the stored electrical energy to the radiation source, the electrical energy storing unit can be, for example, a capacitor or a battery like a thin-film battery. If the processing unit comprises an amplification unit for increasing the generated voltage, before being used by the radiation source for generating the radiation, the amplification unit can comprise, for example, a transformer and/or a voltage multiplier. The voltage multiplier is, for example, a Villard cascade. The voltage multiplier can comprise charging circuitry, for example, a rectifier and a charger. A configuration of a transforming unit 602 with a piezoelectric element 609, electrical energy storing units 651 and an amplification unit 655 is schematically and exemplarily shown in FIG. 14.

In FIG. 14, the transforming unit 602 comprises a piezoelectric element for generating alternating currents (AC) based on received ultrasound waves. A transformer 650 transforms the corresponding AC voltage to a transformed AC voltage. A high voltage cascade 655 comprising capacitors 651 and rectifiers like diodes 652 amplifies the transformed voltage, wherein the capacitors 651 are the electrical energy storing units. Thus, in this embodiment, an integrated electrical energy storing and amplification unit is provided. The amplified high voltage can be provided to the radiation source by connecting the anode of the radiation source to the point 653 and the cathode of the radiation source to the point 654.

The transformer can be a piezoelectric transformer. A piezoelectric transformer uses acoustic coupling between an input and an output of the transformer. For instance, the piezoelectric transformer can comprise a bar of a piezo-ceramic material such as PZT, wherein an input voltage can be applied across a short length of the bar, thereby creating an alternating stress in the bar by the inverse piezoelectric effect and causing the whole bar to vibrate. The vibration frequency is preferentially chosen to be the resonant frequency of the bar and can be, for instance, in the range of 100 kHz to 1 MHz range. A higher output voltage is then generated across another section of the bar by the piezoelectric effect.

The radiation application apparatus 1 further comprises a user interface 73 for allowing a user to, for example, control the motor 67, the catheter navigation unit 66 and/or the ultrasound control unit 71. The user interface 73 is, for example, a combination of an input unit like a keyboard or a mouse and a display unit providing a graphical user interface for allowing a user to control the catheter navigation unit, the motor and/or the ultrasound control unit by inputting corresponding commands.

The radiation application apparatus 1 may further comprise an x-ray fluoroscopy system 68 with an x-ray source 62 and an x-ray detector 64. The x-ray source 62 emits an x-ray beam 65 which traverses the person 3 including the catheter 11. The x-ray beam 65, which has traversed the person 3, is detected by the x-ray detector 64. The x-ray detector 64 generates electrical signals depending on the detected x-ray beam and the electrical signals are used by a fluoroscopy control unit 63 for generating an x-ray projection image. The fluoroscopy control unit 63 is also adapted to control the x-ray source 62 and the x-ray detector 64. The x-ray source 62 and the x-ray detector 64 can be adapted to be rotatable around the person 3 for allowing the x-ray fluoroscopy system 68 to generate x-ray projection images in different directions. The x-ray fluoroscopy system 68 is, for example, a computed tomography fluoroscopy system or a C-arm fluoroscopy system. The fluoroscopy images can be shown on a display 74. The fluoroscopy control unit 63 can be controllable by a user, in order to allow the user to initiate the acquisition of desired fluoroscopy images.

The catheter navigation unit 66 can be adapted to allow a user to navigate the catheter 11 completely by hand or semi-automatically depending on a determined position of the distal end of the catheter 11, wherein the position of the distal end of the catheter 11 can be determined, for example, based on the fluoroscopy images. The position is preferentially only determined while placing the catheter 11 and while placing the radiation source and not during the application of the x-ray radiation by the radiation source within the person.

The catheter 11 comprises preferentially built-in guiding means (not shown in FIG. 1), which can be controlled by the catheter navigation unit 66. The catheter 11 can, for example, be steered and navigated by the use of steering wires, in order to guide the distal end of the catheter 11 to a desired location within the person 3.

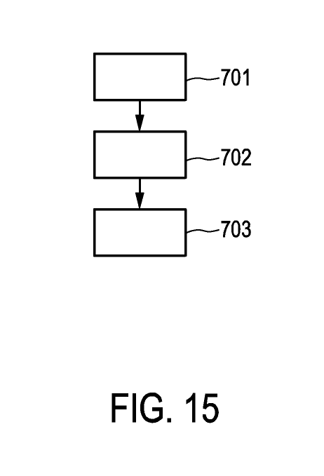

In the following an embodiment of a radiation application method for applying radiation at a location within an object being, in this example, a person will exemplarily be described with reference to a flowchart shown in FIG. 15.

In step 701, the catheter 11 is navigated to a desired location within the person 3, while fluoroscopy images are generated by the x-ray fluoroscopy system 68, which show the actual position of the catheter 11 within the person 3.

In step 702, the transforming unit and the radiation source are introduced into and moved within the catheter 11 by using the motor 67. In step 703, ultrasound energy, i.e. ultrasound waves, are generated by the ultrasound energy generating device, wherein the ultrasound waves are received by the transforming unit, which transforms the ultrasound energy into electrical energy, i.e. into an electrical voltage and an emission current. The electrical voltage is applied to the radiation source such that the radiation source emits radiation at the location within the person, to which the transforming unit and the radiation source have been moved.

The radiation application apparatus is preferentially adapted to perform a brachytherapy. The catheter can therefore be regarded as being a brachytherapy applicator. In other embodiments, also another brachytherapy applicator can be used like a brachytherapy applicator comprising a needle or another interstitial tube. The brachytherapy applicator can also be a balloon applicator, a SAVI-type applicator, et cetera. The brachytherapy applicator can be adapted for being introduced into certain parts of a person. For instance, it can be a vaginal applicator, a breast tumour cavity applicator, et cetera.

The transforming unit is preferentially adapted to transform the ultrasound energy into high voltage for providing electrical energy. The high voltage is preferentially within a range needed for driving an x-ray tube. It is preferentially within the range of 20 kV to 100 kV and further preferred in the range of 40 kV to 100 kV.

The transforming unit can be adapted to provide pulsed electrical energy, wherein the radiation source can be adapted to generate pulsed radiation based on the pulsed electrical energy. In particular, the transforming unit can be adapted to generate a pulsed electric field between the anode and the cathode of the x-ray tube and, thus, a pulsed current, which may be in the kHz to MHz frequency range. In another embodiment, the transforming unit can also be adapted to provide directly continuous electrical energy, if the ultrasound energy is continuous, or via the processing unit, which may comprise a storing unit and rectifiers for generating continuous electrical energy over a certain time, even if the ultrasound energy is pulsed, wherein the radiation source can be adapted to generate continuous radiation based on the continuous electrical energy.

The piezoelectric element is, for example, a ceramic and may comprise one or several of the following materials: PZT, quartz, LiNbO₃, LiTaO₃, GaPO₄, La₃Ga₅SiO₁₄, BaTiO₃, KNbO₃, Na₂WO₃, Ba₂NaNb₅O₅, Pb₂KNb₅O₁₅, NaKNb, BiFeO₃, NaNbO₃. In another embodiment, polymeric piezoelectrica can be used like polyvinylidene fluoride (PVDF), which exhibits significantly higher piezoelectricity than quartz.

Although in the above described embodiments certain arrangements of the transforming units, in particular, of the piezoelectric element, and the radiation source have been shown, in other embodiments also other arrangements can be used for transforming the ultrasound energy into electrical energy, which is applied to the radiation source for generating the radiation to be applied within an object. For instance, a piezoelectric element of the transforming unit and/or the radiation source can also be incorporated in the source applicator such that with introducing the source applicator also the transforming unit and/or the radiation source are introduced in the object.

Although in the above described embodiments several techniques for determining the position of the radiation source and the transforming unit within the object have exemplarily been described, the position of the radiation source and the transforming unit can also be determined in another way. For instance, with the transforming unit and the radiation source a position determining unit and a position sending unit can be introduced into the object. The position determining unit can have a fixed spatial relation with the transforming unit and the radiation source such that the position of the transforming unit and the radiation source is known, if the position of the position determining unit is known. The determined position can be provided to the position sending unit, which sends the determined position to, for example, the ultrasound energy generating device, in order to allow the ultrasound energy generating device to focus the ultrasound waves to the position of the transforming unit. The determined position can also be sent to a display for allowing the display to show the determined position of the radiation source to a user.

The determined position can be sent to the outside of the object wirelessly or via a wired signal connection located, for instance, within a tube like a catheter in which also the radiation source, the transforming unit, the position determining unit and the position sending unit may be present. The position sending unit can be adapted to send the determined position, if ultrasound energy having a certain pulse sequence is received by the transforming unit.

In a further embodiment, the transforming unit can be adapted to charge a miniature rechargeable battery or capacitor, or several miniature batteries, for example, of thin-film type, incorporated in or near a miniature x-ray source. The electrical energy storing unit can be adapted to accumulate the electrical energy and to provide the accumulated energy for applying the radiation, when a predefined voltage has been reached or when a corresponding control signal is received. The control signal can be received, for example, via a control wire connecting an outside control unit with the electrical storing unit within the object. The radiation application apparatus can further comprise a storage state sensor for sensing the storage state of the electrical energy storing unit and a sending unit for sending the sensed storage state to the outside of the object, if the storage state sensor is located within the object. Thus, in an embodiment, the radiation application apparatus may include a sensor function indicating a charge state of a battery and a device transmitting signals indicative of battery charge state ex vivo.

Although in above described embodiments x-ray fluoroscopy images are used for placing the radiation source within the person, in other embodiments also other means can be used for the placement of the radiation source, in particular, for confirming, imaging and/or checking the placement. For instance, ultrasound imaging or other imaging modalities can be used for monitoring the placement of the radiation source. If an ultrasound imaging system is used for monitoring the placement of the radiation source, the ultrasound energy generating device can be integrated with the ultrasound imaging system such that the ultrasound imaging system can provide at least two functions, assisting in placing the radiation source at a desired location within the object and providing ultrasound energy for allowing the transforming unit to generate electrical energy to be applied to the radiation source.

The radiation application apparatus is preferentially adapted to perform electronic brachytherapy, wherein a miniature x-ray tube operating at a modest voltage like 50 kV is used. Advantages of the electronic brachytherapy include that the tube can be turned off and that the radiation energy is relatively low, in particular, compared to standard isotopes used for radioactive brachytherapy, and thus has a short range. This implies that the treatment does not have to be carried out in a standard radiotherapy bunker, but can be performed in interventional x-ray facilities and operation rooms. Therefore, electronic brachytherapy is possible in various departments and outpatient settings and the treatment can be performed by, for example, an interventional radiologist. The healthy tissue of the patient and treatment personnel are spared, and cumbersome isotope logistics and regulations can be disregarded.

In the miniature x-ray tube, the applied voltage gives the energy of the radiation, i.e. the maximum energy of the bremsstrahlung spectrum, and thus the radiation range in tissue. An acceleration voltage of 50 kV gives a mean energy of about 25 keV. The distance to the target tissue is preferentially within the range of 0.5 to 4 cm, requiring radiation energy of about 20 to 50 keV. This means that the acceleration voltage of the miniature tube is preferentially in the range of 40 kV to 100 kV.

The above mentioned ultrasound transmission cables have preferentially a diameter being smaller than 2.0 mm. This diameter is substantially smaller than the diameter of corresponding high voltage cables which would be required without ultrasound high voltage generation as performed by the above described embodiments. Furthermore, less severe risks are associated with ultrasound than with high voltage power transmission in vivo.

The ultrasound waves are preferentially generated ex vivo, for example, by a common ultrasound probe for imaging devices but also other ultrasound actuators could be used. These may be optimized for the coupling to an ultrasound transmission cable to have the highest energy coupling efficiency within the optimized frequency range. Optimized frequency range means the frequency that gives the correct voltage from the piezoelectric element for the electric field of the x-ray source. The piezoelectric element can be used for charging a thin-film battery/capacitor or for directly generating an electric field between cathode and anode of the miniature source. In this way, both high voltage cables and the cumbersome contacts from high voltage leads to the radiation source electrodes, which are very time-consuming to mount, are eliminated. This opens up the possibility of simple design of miniature sources for spectra up to 100 kVp which would otherwise be limited by the use of high voltage cables due to necessary isolation thicknesses. If, in another embodiment, an integrated high-voltage generator including a piezoelectric element and an ultrasound actuator is made by MEMS technology, only the low voltage and mid to high frequency signal is transmitted via a thin and flexible cable.

The above described embodiments use temporarily inserted miniature x-ray sources. However, in other embodiment the radiation source together with the transforming unit can be permanently implanted, wherein the ultrasound energy can be provided from ex vivo to the permanently in vivo implanted transforming unit. In this case, the transforming unit and radiation source can be intraoperatively or percutaneously placed using, for instance, a syringe, in, for instance, a tumor or a tumor cavity.

The radiation application apparatus can be adapted for the treatment of, for example, prostate, breast, rectum, vaginal, liver, kidney, esophagus, lung, skin, head and neck cancer.

Although in the embodiment described above with reference to FIG. 1 the ultrasound energy generating device is located on the person, it can also be arranged at another location outside the person, wherein optionally the ultrasound energy generating device can be connected with an ultrasound transferring unit for transferring the ultrasound energy into the person. Moreover, the ultrasound energy generating device can also be arranged close to the transforming unit within the person.

Although in the above described embodiments the radiation source comprises certain cathodes and anodes, in other embodiments the radiation source can also comprise another cathode and/or anode. The cathode may be, for example, a thermal filament, a field emitting cathode, a Shottky cathode, a piezo- or ferroelectric cathode, or a combination thereof. The anode may be of reflection or transmission type. Optionally, a third electrode, a gate, can be incorporated, for example, in form of a floating or cathode potential electrostatic lens. Moreover, between the cathode and the anode an intermediate dielectric can be provided. Furthermore, the anodes can be transmission type anodes, reflection type anodes or a mixture of a transmission type and a reflection type anode.

Although in the above described embodiments the radiation source is an x-ray tube, in other embodiments also other radiation sources can be used. For instance, the radiation source can be a radiation source generating light within another wavelength range, for instance, in the visible wavelength range. The radiation source can also be a lasing device.

Although in the above described embodiments certain techniques for moving the radiation source and the transforming unit within the object have been described, the radiation source and the transforming unit can also be moved by using another technique. For instance, they can be moved by using the technique of moving an ultrasound imaging probe within a natural cavity as known from, for example, endovaginal, endorectal, or transesophageal ultrasound imaging.

Other variations to the disclosed embodiments can be understood and effected by those skilled in the art in practicing the claimed invention, from a study of the drawings, the disclosure, and the appended claims.

The figures are schematically only. For instance, they are not to scale, i.e., for example, the electrodes are thinner than shown in the figures, and the moving wire can be arranged centrally or at an off center position on the respective housing.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A radiation application apparatus for applying radiation at a location within an object, the radiation application apparatus (1) comprising:
a transforming unit (2; 102, 112; 202; 302; 402; 502) for being arranged within the object (3) at the location and for transforming ultrasound energy to electrical energy,
a radiation source (4; 104; 204; 304; 404; 504) for being arranged within the object (3) and for generating radiation (5) to be applied at the location within the object (3), wherein the radiation source (4; 104; 204; 304; 404; 504) is driven by the electrical energy,
an ultrasound energy generating device (31; 531) for generating ultrasound energy to be transformed by the transforming unit (2; 102, 112; 202; 302; 402; 502) to electrical energy.

2. The radiation application apparatus as defined in claim 1, wherein the radiation source (4; 104; 204; 304; 404; 504) is an x-ray source for generating x-rays (5) as radiation, when the electrical energy is applied to the x-ray source.

3. The radiation application apparatus as defined in claim 1, wherein the transforming unit (2; 102, 112; 202; 302; 402; 502) comprises a piezoelectric element for transforming the ultrasound energy into electrical energy.

4. The radiation application apparatus as defined in claim 3, wherein the transforming unit (302) comprises several piezoelectric elements (309, 313, 324, 325) for transforming the ultrasound energy into voltage for providing electrical energy, wherein the piezoelectric elements (309, 313, 324, 325) are arranged such that the voltages of the several piezoelectric elements are combined to a combined voltage being larger than each voltage produced by a respective single piezoelectric element (309, 313, 324, 325).

5. The radiation application apparatus as defined in claim 3, wherein the radiation source (4) is an x-ray source for generating x-rays as radiation, when the electrical energy is applied to the x-ray source (4), and wherein the piezoelectric element (9) is integrated with the anode (6) or the cathode (7).

6. The radiation application apparatus as defined in claim 1, wherein the object is a person and wherein the transforming unit (2; 102, 112; 202; 302; 402; 502) and the radiation source (4; 104; 204; 304; 404; 504) are configured to be arrangable within the person for applying the radiation (5) at the location within the person (3) by using an applicator (11).

7. The radiation application apparatus as defined in claim 1, wherein the radiation application apparatus further comprises an electrical energy storing unit (651) for storing the electrical energy and for providing the stored electrical energy to the radiation source.

8. The radiation application apparatus as defined in claim 1, wherein the transforming unit (602) is adapted to generate a voltage for providing the electrical energy, wherein the radiation application apparatus further comprises an amplification unit (655) for increasing the generated voltage, before being used by the radiation source for generating the radiation.

9. (canceled)

10. The radiation application apparatus as defined in claim 14, wherein the ultrasound energy generating device (31) is adapted to:
send the ultrasound energy to the object (3),
receive reflected ultrasound energy from the object (3),
determine the position of the transforming unit (2; 102, 112; 202; 302) within the object (3) from the received reflected ultrasound energy, and
focus the send ultrasound energy onto the determined position of the transforming unit (2; 102, 112; 202; 302).

11. The radiation application apparatus as defined in claim 19, wherein the object is a person (3) and wherein the transforming unit (502), the radiation source (504), and the ultrasound energy generating device (531) are configured to be arrangable within the person (3) for applying the radiation (5) at the location within the person (3) by using an applicator (11).

12. The radiation application apparatus as defined in claim 1, wherein the radiation application apparatus (1) further comprises an ultrasound transferring unit (430) for transferring ultrasound waves from the outside of the object (3) to the transforming unit (402).

13. The radiation application apparatus as defined in claim 1, wherein the radiation application apparatus (1) is adapted to be used for interventional radiotherapy, wherein the radiation is provided by x-rays (5) which are applied to the object.

14. (canceled)

15. (canceled)
